(19) United States
(12) Patent Application Publication
(10) Pub. No.: US 2017/0137777 A1
(43) Pub. Date: May 18, 2017

(54) IN VITRO CELLULAR ANALYSIS

(71) Applicants: Morteza Mahmoudi, Tehran (IR); Shahriar Sharifi, Tehran (IR); Mohammad Raoufi, Tehran (IR)

(72) Inventors: Morteza Mahmoudi, Tehran (IR); Shahriar Sharifi, Tehran (IR); Mohammad Raoufi, Tehran (IR)

(21) Appl. No.: 15/420,060

(22) Filed: Jan. 30, 2017

Related U.S. Application Data
(60) Provisional application No. 62/289,272, filed on Jan. 31, 2016.

(51) Int. Cl. C12N 5/00; G01N 33/50

(52) U.S. Cl. CPC C12N 5/0062; C12N 5/0068; G01N 33/5008; G01N 33/5014; C12N 2513/00

(57) ABSTRACT

A plurality of initial biological cells is provided, and the initial biological are cultured cells on a pseudo-3D substrate that has a cell-resembling surface to form a plurality of cultured cells. The plurality of cultured cells is subjected to a cell analysis assay. Optionally, the response of the cultured cells to the cell analysis assays is evaluated.

### CROSS REFERENCE TO RELATED APPLICATION

The present application claims priority from pending U.S. Provisional Patent Application Ser. No. 62/289,272, filed Jan. 31, 2016, entitled “3D CELL CULTURE AND IN VITRO TOXICITY TESTING METHODS”, which is incorporated by reference herein in its entirety.

### TECHNICAL FIELD

The present application generally relates to biological cells behavior investigations, particularly, a method for accurate cellular analysis and more particularly, a method for accurate cellular analysis of biological cells treated by a test agent using a pseudo-3D substrate with a cell-resembling surface.

### BACKGROUND

Static well plate or layer systems are known techniques of cellular assays for in vitro study. These assays are straightforward and provide a convenient testing environment for monitoring cell behavior and measuring chemicals in the cell culture medium as well as in the cell. In these cell studies, a monolayer of single or multiple cell types that are either freshly isolated from human or animal tissues or are already established immortalized cell lines are used. Cell culture studies can be performed on two-dimensional (2-D) flat surfaces, for example, tissue culture well plates, tissue culture flasks, Petri-dishes and well microplates.

All cells in a body, though, are situated in a 3D environment. That is crucial for their functions, including proliferation and metabolic activity. However, one example problem associated with 2D cell culture as a non-real representative of in vivo models is that there may be a large incongruity in results between in vitro 2D-cell-culture and animal models. For instance, recent studies on in vitro toxicity testing of quantum dots, super paramagnetic iron oxide (SPIO) nanoparticles (NPs), carbon nanotubes, and fullerenes using in vitro 2D cell culture showed high cytotoxic effects. However, tested in animal models, adverse effects were not observed. This is one example showing a need for a suitable 3D cell model for cellular assays.

One known 3D cell culture technique for evaluating cellular assays in 3D systems utilizes spheroids (cell aggregates) by either using a scaffold/matrix or a scaffold-free arrangement. Spheroids (cell aggregates) represent a simple 3D system since no scaffold or supporting material is required for 3D cell growth. Known scaffold-free techniques to generate spheroids include forced-floating techniques such as magnetic fields to suspend cells, non-adhesive microwells, and agitation-based approaches such as rotation cultures, hanging drop or some combination of these approaches. However, for various reasons cellular assays using developed 3D cultures can be more complicated than using 2D cultures.

Hence, there is a need for accurate methods of cellular analysis using a cell culture support with a biomimetic surface. There is also a need for methods and 3D cell culture media to provide efficient ways and natural environments for cellular investigations.

### SUMMARY

Disclosed methods include a method that can provide, in one general aspect, cellular analysis of a number of biological cells. The method may include steps of providing a plurality of initial biological cells; forming a plurality of cultured cells by a process comprising culturing the initial biological cells on a cell-resembling surface of a pseudo-3D substrate; and obtaining a response of the cultured cells to at least one cell analysis assay.

In an implementation, the method can further include evaluating the response of the cultured cells to at least one of the at least one cell analysis assays.

The above general aspect may include one or more of the following features. In an implementation, providing the plurality of initial biological cells can include supplying a plurality of biological cells from a cell bank or from a biopsy operation, or from a combination of a cell bank and a biopsy operation; and culturing the plurality of biological cells to provide a plurality of biological initial cells.

In an implementation, the cell-resembling surface can include a surface having a structure resembling a surface structure of one cell, or resembling a surface structure of an association of cells, or resembling a combination of a surface structure of one cell and the surface structure of an association of cells

According to an implementation, the cell analysis assay may be at least one of the assays including gene regulation assays, gene expression assays, protein expression assays, cell proliferation assays, cell-cycle assays, cell viability assays, cell cytotoxicity assays, cell morphology assays, cell cytoskeleton assays, apoptosis assays, cell adhesion and signaling assays, cell motility assays, tissue architecture assays, co-cultures assays, biochemical assays, molecular biological assays or combinations thereof.

In an implementation, the method can further comprise fabricating the cell-resembling surface of the pseudo-3D substrate, and in an aspect, the fabricating can include forming a cell-resembling mold by a process that can include recording the surface profile of the initial biological cells onto a first substrate; and forming cell-resembling surface by a process that can include replicating the surface profile of cells from the cell-resembling mold onto a second substrate

Disclosed methods can include a method that can provide, according to one or more implementations, cellular analysis of the plurality of biological cells treated by a test agent, and steps can include comprising: providing a plurality of initial biological cells; forming a plurality of cultured cells by a process that can include culturing the initial biological cells on a cell resembling surface of a pseudo-3D substrate; treating the cultured cells with at least one test agent; and obtaining a response of the treated cells to at least one cell analysis assay.

In an implementation, the treating the cultured cells with the test agent can include: adding the test agent on the cultured cells on the pseudo-3D substrate; and maintaining the pseudo-3D substrate including the cultured cells with the added test agent in an incubator.

The cultured cells may be treated with a test agent via a two-step method that can include: adding the test agent on the cultured cells on the pseudo-3D substrate; and maintaining the pseudo-3D substrate including the cultured cells with the added test agent in an incubator to achieve cells treatment.

In some implementations, the pseudo-3D substrate with a cell-resembling surface may be fabricated via a method that can include: recording the surface profile of the initial cells onto a first substrate to form a cell-resembling mold; and replicating the surface profile of cells from the cell-resembling mold onto a second substrate to form the pseudo-3D substrate with a cell-resembling surface. The cell-resembling mold may be a pseudo-3D substrate having a cell-resembling surface.

In an implementation, the first substrate may include a crosslinkable polymer with acrylates double bonds or with epoxy bonds and may made from silicon, or silicon resins, or Poly(dimethylsiloxane) (PDMS). In an implementation, the second substrate may include non-degradable polymers, for example, a polystyrene (PS) or a polycarbonate substrate.

In some implementations, recording the surface profile of cells can include, for example, cells imprinting, 3D imprinting, 4D printing, casting, scanning, AFM imaging, confocal laser microscopy (CLSM).

In some implementations, replicating the surface profile of cells from the cell-resembling mold onto a second substrate can be performed or assisted through a casting process, or an imaging technique, or photo curing, or hot embossing, or 3D printing, or rapid prototyping, or micro rapid prototyping using a SLA technique.

Example features of disclosed methods are providing an imitating of nanoscale and macro scale detail surface morphology of the plasma membrane, that can further provide the required topographical cell fingerprints to induce differentiation of stem cell toward desired cell lines.

### BRIEF DESCRIPTION OF THE DRAWINGS

FIG. 1 illustrates an example method for cellular analysis of biological cells, pursuant to the teachings of the present disclosure.

FIG. 2 illustrates an example method for cellular analysis of biological cells treated by a test agent, according to exemplary implementations of the present disclosure.

FIG. 3A-3B illustrates a schematic of one example of a recording process of the surface profile of example initial cells onto an exemplary substrate to form an example of a cell-resembling mold, pursuant to the teachings of the present disclosure.

FIG. 3C-3E illustrates a schematic of one example of a replicating process of the surface profile of cells from an example cell-resembling mold onto an example substrate to form an example of a pseudo-3D substrate having a cell-resembling surface, pursuant to the teachings of the present disclosure.

FIG. 4A shows uptake amount of SPIOs by HeLa cells cultured on various exemplary substrates having pseudo-3D cell-resembling surfaces as described in more detail in connection with EXAMPLE 5.

FIG. 4B shows uptake amount of SPIOs by 3T6 cells cultured on various exemplary substrates having pseudo-3D cell-resembling surfaces as described in more detail in connection with EXAMPLE 5.

FIG. 5A shows the cell viability (%) determined by a mitochondria activity assay for example HeLa cells cultured on various exemplary substrates having pseudo-3D cell-resembling surfaces as described in more detail in connection with EXAMPLE 5.

FIG. 5B shows the cell viability (%) determined by a mitochondria activity assay for example 3T6 cells cultured on various exemplary substrates having pseudo-3D cell-resembling surfaces as described in more detail in connection with EXAMPLE 5.

FIG. 6 shows examples of Immunofluorescence microscopy images illustrating co-localization of focal adhesion kinase (FAK) with polymerized actin microfilaments in HeLa cells cultured on collagen-coated coverslips or on HeLa cells-resembling substrate and incubated with non-fluorescent nanoparticles at a concentration of about 500 μM.

### DETAILED DESCRIPTION

2D cell culture on a flat substrate may not be considered an appropriate means for demonstrating or reliably determining in vivo cellular responses. Among reasons can be the high dependency of phenotype and function of individual cells on intricate interaction of cells with 3D-organized extracellular matrix (ECM) proteins, as well as their interaction with neighboring cells. In addition, primary cells generally have a limited lifespan, and generally do not have a stable phenotype. Although established cell lines are more stable, they do not present genuine tissue specific function. Therefore, 2D may not accurately predict in vivo toxicity and other biological effects, due to the absence of key 3D organization.

However, there are shortcomings associated with various known concepts of 3D cell culture. Such shortcomings can be particularly associated with spheroids 3D systems. For instance, small-sized spheroids may not display the complexity of real tissue, while larger spheroids can result in reduced viability of the cells, and can incur necrotic core formation due to limitations in diffusion of oxygen and nutrients. In one example implementation, an ideal size for spheroids can range from about 100 μm to about 600 μm for each cell line. This can necessitate separate investigations, since the spheroid critical size can be different depending on cell type and cell packing density. Another shortcoming associated with 3D cell culturing techniques can be a difficulty in counting cells in 3D structures. For example, when the culture is particulate it may be difficult to take a representative portion of a 3D spheroid. Still another shortcoming associated with 3D cell culturing technique is that cells in mature 3D cultures can grow significantly slower than their 2D counterparts.

Notwithstanding the above shortcomings, the concept of 3D cell culture and spheroid formation has been used for drug and soluble molecules as test agents. In more specific cases, such as evaluation of the toxicity of nanoparticles on cells using spheroids with both scaffolding and scaffolding-free technique there are additional challenges. With advancement in the nanotoxicology field, it is known that despite similarities, further considerations need to take be taken into account when a common toxicological testing of drugs or soluble compounds is extended to nanoparticles toxicity, including considerations that relate to conflicting data regarding the toxicity of nanoparticles. For instance, factors including protein coronas, cell types, local temperatures, nanoparticles dosage inside the cell, cell density and protein source are now known causes for such divergence. Without subscribing to any particular theory, it is believed that interference of nanoparticles with conventional nano-toxicity assays may be another factor that should be considered in, or prior to applying nanotoxicology test.

In cell spheroids, only a subset of cells may be exposed to the nanoparticles, because the ECM and the dense cell packing may hinder nanoparticles penetration to the spheroid core. Moreover, the penetration of nanoparticles in spheroids may be limited by nanoparticles-cell interaction. Therefore, despite the fact that 3D cell culture has provided, for example, some insight into the interaction of nanoparticles with ECM components, usefulness of such systems is constrained by the limited information they may provide about interactions of nanoparticles with cells.

Accordingly, a method for in vitro cellular analyses for accurate in vivo tests simulation is disclosed herein, which can include among other features a pseudo-3D substrate having a cell-resembling surface designed and fabricated for cellular analysis.

In an aspect, the present disclosure describes a method for cellular analysis of biological cells, for example biological cells treated by a test agent, for example, drugs or nanoparticles. In some aspects, the biological cells may be cultured on pseudo-3D substrates having cell-resembling surfaces in order to enable the disclosed method to be an accurate analysis method for in vitro applications.

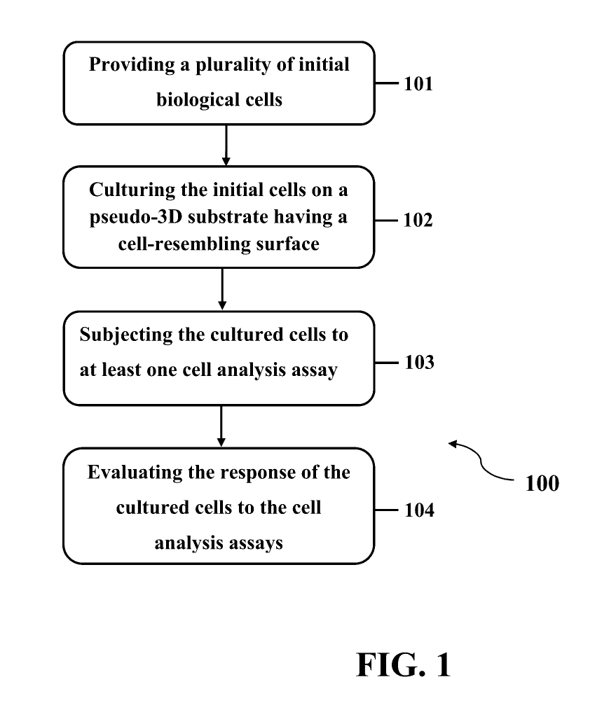

FIG. 1 illustrates steps in one implementation of a method 100 that can provide, according to one or more aspects, cellular analysis of a plurality of biological cells. The FIG. 1 implementation of the method 100 can include steps of: providing a plurality of initial biological cells (step 101); culturing the initial cells on a pseudo-3D substrate having a cell-resembling surface to form a plurality of cultured cells (step 102); subjecting the cultured cells to at least one cell analysis assay (step 103); and evaluating the response of the cultured cells to the cell analysis assays (step 104).

Referring to FIG. 1, in an implementation, providing a plurality of initial cells in step 101 can include supplying a plurality of biological cells and culturing the plurality of biological cells in a cell culture medium at a controlled set of conditions to provide a plurality of initial cells. In an implementation, the initial biological cells may be prepared, for example, by seeding a plurality of biological cells in a range of about 1000 cells to about 1,000,000 cells or higher into a culture media and culturing them.

In one implementation, the biological cells can be supplied or obtained from a cell bank or through a biopsy operation. The biological cells can be for example, stem cells, adipose-derived stem cells (ADSC), chondrocytes, tenocytes, HeLa, 3T6, cardiomyocytes, myoblasts, smooth muscle cells, neuron-cells, immune cells, and combinations thereof.

In one implementation, the biological cells can be cultured in a cell culture media, for example, Roswell Park Memorial Institute (RPMI) medium or DMEM. In an aspect, the cell culture media can be supplemented with a protein source, for example, Fetal bovine serum (FBS) or human serum/plasma and penicillin/streptomycin. Furthermore, the biological cells may be cultured at a controlled set of conditions, for example, at a controlled temperature, for example at about 37° C. and a controlled humidity, for example, at about 5% CO₂.

Referring to FIG. 1, at step 102, the initial cells, obtained in step 101, can be cultured on a pseudo-3D substrate having a cell-resembling surface to form a plurality of cultured cells. Culturing on a pseudo-3D substrate may provide a simulated in vitro cell culturing similar to an in vivo cell environment, because of the similarities of the surface structure of the culturing substrate to exemplary initial biological cells. To achieve a more accurate cellular analysis or, more generally, a more accurate cell behavior analysis of, for example, cellular uptakes, intracellular pathways and cell functions, the initial cells can be cultured on a pseudo-3D substrate with a same cell-resembling surface with the initial cells. For example, in HeLa cells analysis, a substrate having a 3D surface structure resembling the HeLa cells 3D structure can be used.

As used herein, a “cell-resembling surface” can refer to a surface having pseudo-3D shape, or pseudo-3D topography, or pseudo-3D imprinted structure, or 3D shape, or 3D topography, or 3D imprinted structure, such as one biological cell or an association of cells resembling the surface structure of one cell or an association of cells.

Referring to FIG. 1, at step 103 at least one cell analysis assay can be applied to the cultured cells obtained in step 102. The cell analysis assay may include, for example, gene regulation assays, gene expression assays, protein expression assays, cell proliferation assays, cell-cycle assays, cell viability assays, cell cytotoxicity assays, cell morphology assays, cell cytoskeleton assays, apoptosis assays, cell adhesion and signaling assays, cell motility assays, tissue architecture assays, co-cultures assays, biochemical assays and molecular biological assays.

In one implementation, cell viability assays can include mitochondria activity assay or a MTT assay, MTS assay, XTT assay, WST assay, Comet assay, TUNEL assay, Annexin V assay, etc.

Referring to FIG. 1, at step 104, the response of the cultured cells to the cell analysis assays resulted in step 103 may be evaluated to determine the cultured cells behavior or functions. In some implementations, the evaluation may include detecting and analyzing for example, the percent of cells viability, the cellular functions, etc.

In some implementations, the disclosed method 100 can be used to analyze the effects of a test agent on a plurality of biological cells. For example, method 100 can be used to analyze the toxicity caused by a test agent while treating the biological cells with the test agent. For instance, the method 100 can be used for cellular analysis of a plurality of cells treated by nanostructures or nanoparticles. In some specific examples, the method 100 can be applied to analyze and compare the effects on a plurality of cells caused by nanostructures or nanoparticles of same composition but with different physicochemical properties including size, shape, surface properties, etc.

As used herein, “test agent” refers to any substance that may be evaluated for its ability to diagnose, cure, mitigate, treat, or prevent disease in a subject, or is intended to alter the structure or function of the body or cells of a subject. Test agents include, but are not limited to drugs, chemical compounds, biologic agents, proteins, peptides, nucleic acids, lipids, polysaccharides, supplements, diagnostic agents, immune modulators, nanostructures and nanoparticles.

In one implementation, a test agent may be a nanostructured cell treating agent, for example, nanoparticles (NPs), quantum dots, carbon nanotubes, fullerenes, and combinations thereof.

In some implementations, a test agent may be for example, organic nanoparticles or nanostructures, polymeric nanoparticles or nanostructures and inorganic nanoparticles or nanostructures.

In another implementation, a test agent may be for example, nanoparticles including or made from metallic nanoparticles, for example, nanoparticles including iron, silver, gold, cadmium, titanium, etc. In another example, a test agent can be nanoparticles including or made from metal oxides, for example, iron oxide nanoparticles, super paramagnetic iron oxide (SPIO) nanoparticles, titanium oxide nanoparticles, etc.

As used herein, the term “toxicity” or “cytotoxicity” refers to any unwanted effect on human cells, animal cells or corresponding tissues caused by a test agent, or test agent used in combination with other pharmaceuticals.

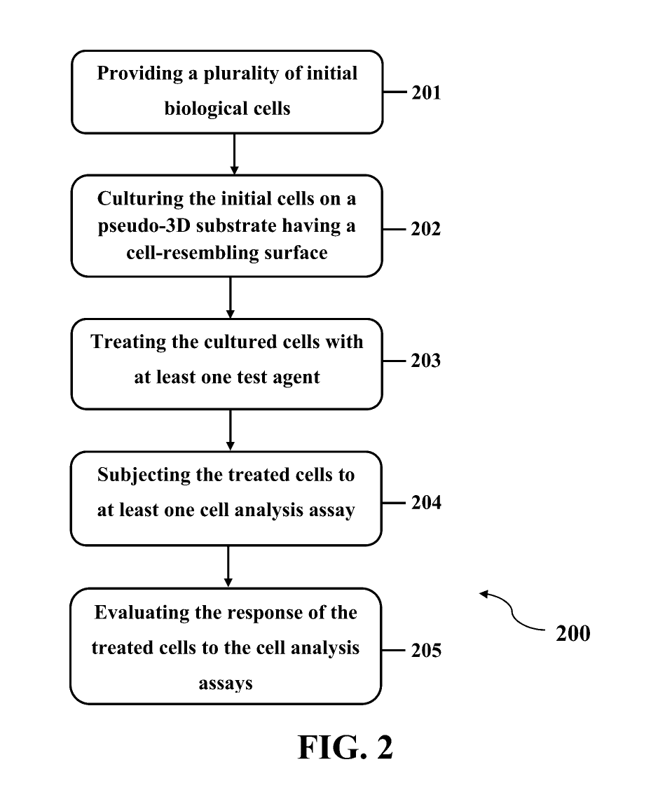

FIG. 2 illustrates an implementation of an example method 200 for cellular analysis of a plurality of biological cells treated by at least one test agent, according to one or more features of the present disclosure. As illustrated by FIG. 2, in an implementation, method 200 may include steps of: providing a plurality of initial biological cells (step 201); culturing the initial cells on a pseudo-3D substrate having a cell-resembling surface to form a plurality of cultured cells (step 202); treating the cultured cells with at least one test agent (step 203); subjecting the treated cells to at least one cell analysis assay (step 204); and evaluating the response of the treated cells to the cell analysis assays (step 205). Accordingly, the effects of the test agents on biological cells, for example, toxicity of the test agent may be determined by analyzing the evaluated responses.

Referring to FIG. 2, a plurality of initial biological cells may be provided in step 201 as described in detail in step 101 of method 100 hereinabove.

Moving on to step 202, the initial cells, obtained in step 201, may be cultured on a pseudo-3D substrate having a cell-resembling surface to form a plurality of cultured cells as described in step 102 of method 100 hereinabove.

Moving on to step 203, in an implementation the cultured cells in step 202 may be treated within the culture by at least one test agent that may be added to the cultured cells on the cell-resembling surface of the pseudo-3D substrate.

In one implementation, treating of the cultured cells with a specific amount of a test agent may be carried out via two steps of: adding a test agent on the cultured cells obtained in step 202; and maintaining the pseudo-3D substrate including the cultured cells with the added test agent in an incubator to achieve a complete cell treatment. In an implementation, the pseudo-3D substrate may be maintained in an incubator for a specific time interval, for example, between about 1 minute and about few months depending on the nature of the test agent and biological cells be used in the method 200.

Moving on to step 204, in an implementation, the cultured cells obtained in step 203 can be subjected to at least one cell analysis assay. The cell analysis assay may include, for example, gene regulation assays, gene expression assays, protein expression assays, cell proliferation assays, cell-cycle assays, cell viability assays, cell cytotoxicity assays, cell morphology assays, cell cytoskeleton assays, apoptosis assays, cell adhesion and signaling assays, cell motility assays, tissue architecture assays, co-cultures assays, biochemical assays and molecular biological assays.

In one implementation, cell viability assays may include mitochondria activity assay or a MTT assay, MTS assay, XTT assay, WST assay, Comet assay, TUNEL assay, Annexin V assay, etc.

Moving on to step 205, in an implementation, the response of the cultured cells to the cell analysis assays resulted in step 204 may be evaluated to determine the cultured cells behavior or functions changed or affected after treatment with a test agent.

In some implementations, the evaluation can include detecting and analyzing for example, the amount of a cellular treating agent uptake by the cultured cells or the percent of cells viability after a cellular treatment.

In another aspect of the present disclosure, a pseudo-3D substrate having a cell-resembling surface may be fabricated via a method including the steps of: recording the surface profile of the initial cells onto a first substrate to form a cell-resembling mold; and replicating the surface profile of cells from the cell-resembling mold onto a second substrate to form the pseudo-3D substrate with a cell-resembling surface. The obtained cell-resembling mold or the obtained pseudo-3D substrate with the cell-resembling surface can be used for in vitro cellular analyses method, for example, the methods described hereinabove.

In some implementations, the first substrate may be made of a crosslinkable polymer with acrylates double bonds or with epoxy bonds and the first substrate may be selected from the group consisting of silicon, silicon resins and poly(dimethylsiloxane) (PDMS).

In some implementations, the cell-resembling mold may have a cell-resembling surface and may be regarded and used as a pseudo-3D substrate having a cell-resembling surface in cellular analyses or cell behavior analyses, for example, cell toxicity analyses.

In one implementation, the surface profile of the initial cells can be recorded onto the first substrate by any from among various techniques such as, for example, cells imprinting, or 3D imprinting, or 4D printing, or casting, or scanning, or AFM imaging, or confocal laser microscopy (CLSM) techniques.

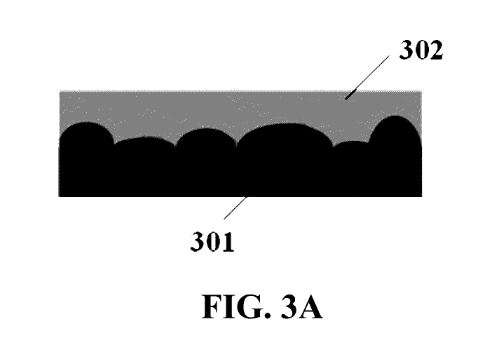

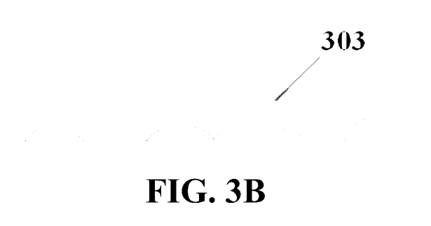

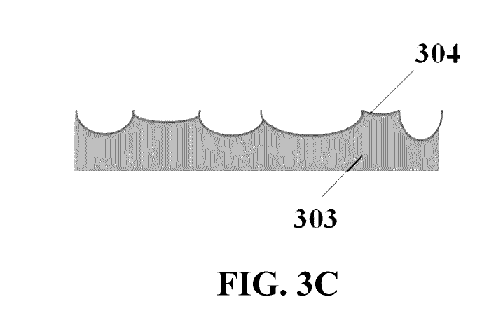

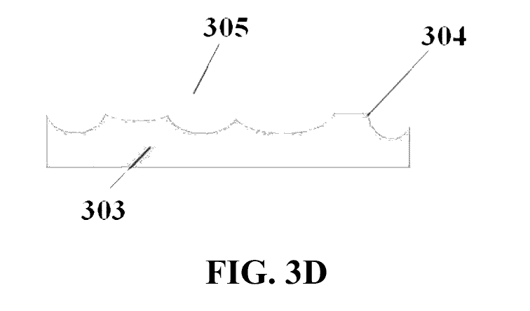

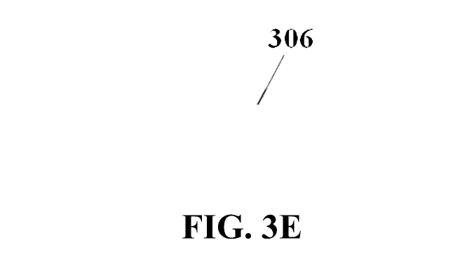

FIGS. 3A and 3B illustrate a schematic of one example of a casting or cell-imprinting process or technique that can be used as a recording process of the surface profile of example initial cells onto an exemplary substrate to form an example of a cell-resembling mold.

Referring to FIGS. 3A and 3B, in some implementations of the illustrated example, the surface profile of the initial cells can be recorded onto the first substrate via a casting process that can include: covering one or an association of initial cells 301 with a first substrate 302 (FIG. 3A), and then, removing the cells 301 from the substrate 302 to obtain a mold 303 having a cell-resembling surface (FIG. 3B).

In some implementations, the obtained mold 303 imprinted with cell surface pattern can be directly used in either a negative or reverse position for a cell culturing operation. Further, in an aspect, the mold 303 can be used in a negative or reverse position or for replicating the surface profile of cells onto a second substrate to transfer the cell topography pattern to the surface of any other substrate.

In another implementation, the surface profile of a single cell can be recorded by an AFM technique. In an aspect, AFM may be employed in both non-contact and contact procedures. According to an implementation, the original surface structure of cells can be coated with a metal before an AFM measurement. In this way, data with a very high spatial resolution may often be obtained.

In some implementations, the surface profile of the initial cells may be replicated from the cell-resembling mold onto a second substrate assisted by a replicating process, for example, casting process, or an imaging technique, or photo curing, or hot embossing, or 3D printing, or rapid prototyping, or micro rapid prototyping using a SLA technique.

In one implementation, the second substrate can be made from for example, a non-degradable polymer. The non-degradable polymer can be for example, polystyrene (PS) or polycarbonate, which are also used in fabricating 2D multi-well cell or tissue culture plates and flasks.

In one implementation, the surface profile of initial cells can be replicated from the cell-resembling mold onto a second substrate using a hot embossing technique. According to one or more implementations, the hot embossing technique can include: depositing a layer of Gold (Au) on the cell-resembling mold, depositing a layer of Nickel (Ni) on the deposited Au layer, removing the mold from the Ni layer to form a Ni stamp having cells structure, hot embossing the Ni stamp to the second substrate; and removing the Ni stamp from the second substrate to obtain the pseudo-3D substrate with a cell-resembling surface. Accordingly, the Gold layer may be deposited using a sputtering technique and the Ni layer may be deposited using an electroforming or electroplating technique.

FIGS. 3C-3E illustrate a schematic of one example of a replicating process of the surface profile of cells from an example cell-resembling mold 303 onto an example second substrate to form an example of a pseudo-3D substrate having a cell-resembling surface.

Referring to FIG. 3C, a layer of Gold (Au) 304 may be deposited on the cell-resembling mold 303 in a negative or reverse position. After that, a layer of Nickel (Ni) 305 may be deposited on the deposited Au layer 304 as shown in FIG. 3D. Then, the mold 303 may be removed from the Nickel layer 305 to form a Nickel stamp 306 having cells structure (FIG. 3E). In an implementation, the Nickel stamp 306 can be used to replicate the surface profile of initial cells to a second substrate, for example, by hot embossing the Nickel stamp to the second substrate. Consequently, a pseudo-3D substrate with a cell-resembling surface may be obtained by removing the Nickel stamp from the second substrate.

In some implementations, cell surface replicate models can be directly created using a nano-scribe micro 3D printer.

In another implementation, replication of cell surface profile can be done directly by using micro rapid prototyping technique. By using this approach, surface replica may be directly made using imaging data obtained by confocal or AFM imaging. This process may eliminate the need for intermediate moldings process. Images obtained from AFM or confocal may be converted to the STL file format prior to production on the rapid prototyping machines.

### EXAMPLES

### Example 1: Providing Initial Biological Cells: Isolation and Culture of Biological Cells

In this example, the mouse fibroblast line 3T6 and HeLa cells (human cervix carcinoma cells) were purchased from the American Type Culture Collection (ATCC, Rockville, Md.) and used throughout the work. HeLa cells were grown in a RPMI medium, supplemented with about 10% fetal calf serum and about 1% penicillin/streptomycin. 3T6 cells were grown in a high glucose DMEM, supplemented with about 10% fetal calf serum, about 2 mM l-glutamine and about 10,000 U/ml of penicillin-streptomycin. Growth temperature was about 37° C. in controlled humidity at about 5% CO₂. Media were changed about 4 hours after plating and subsequently every about 48 hours. One day prior to experiments, the cells were detached in trypsin-EDTA and grown in complete medium in 48-well plates at about 10⁴ cells per well.

Chondrocytes were isolated from cartilage slices and tenocytes were isolated from Achilles tendons of New Zealand white rabbits. Adipose-derived stem cells (ADSC) cells were harvested from the upper part of the intestine and digested in collagenase type I (0.02 mg/mL) for about 1 hour at a temperature of about 37° C. A total of about 3×10³ cells were seeded on a polystyrene cell culture plate for about 24 hours in Dulbecco's Modified Eagle's Medium (DMEM)/Ham's F12 supplemented with about 10% fetal bovine serum (FBS), about 100 U/mL penicillin, and about 100 μg/mL streptomycin. Growth temperature was about 37° C. in controlled humidity at about 5% CO₂. Media were changed about 4 hours after plating and subsequently every about 48 hours. Chondrocytes were grown for one week till they acquired mature spherical shaped morphology.

Production of Pseudo-3D Substrates

### Example 2: Recording the Surface Profile of Biological Cells: A Cell-Imprinting Technique

In this example, pseudo-3D substrates were fabricated using a cell-imprinting method. A silicon substrate (Poly (dimethylsiloxane) (PDMS)) was used to fabricate the cell-imprinted substrates. The PDMS and a curing agent (super paramagnetic iron oxide nanoparticles) were first mixed with about 10:1 ratio and then heated for about 30 minutes to about 35 minutes at a temperature of about 45° C. with a digitally controlled microplate heater to obtain an elastomer solution of the cured PDMS. The biological cells, including Chondrocytes, Tenocytes, HeLa, 3T6 and ADSC were fixed with about 4% glutaraldehyde and the cell medium was completely removed from the plates. The elastomer solution of the cured PDMS substrate was cooled to about 37° C. and then was poured onto each of the cell cultured samples and incubated at about 37° C. for about 24 hours to about 48 hours.

Subsequently, the cured PDMS substrate was peeled from the cell culture plates in all experiments. This step was followed by numerous washing procedures of the PDMS substrates using boiled water and about 1 M NaOH solution to remove remaining cells/debris and other chemicals from the substrates. The obtained PDMS mold imprinted through this example, which have cell-resembling surfaces corresponding to Chondrocytes, Tenocytes, HeLa, 3T6 and ADSC cells, may be used directly for cell culturing or used to replicate the imprinted surface profile of cells onto other substrates.

### Example 3: Replicating the Surface Profile of Cells on a Substrate: A Hot Embossing Technique

PDMS mold imprinted with cell surface pattern, for example which was obtained in EXAMPLE 2, may be used to transfer the cell topography pattern to the surface of any other substrate. In this example, PDMS molds imprinted with cell surface pattern were used to replicate the surface profile of cells on polystyrene substrates to prepare polystyrene substrates surfaces with cell-resembling pattern.

In this regards, firstly, negative (inverse) PDMS molds were seeded with a layer of Au using a sputter coater until the PDMS mold became electrically conductive. Then, an electroforming process was done on the Au-covered mold with a Nickel sulfamate solution in an electroforming bath at about 55° C. with a pH of about 3.7. The current density was kept at about 1 mA·cm² for about 6 hours, and then was increased to an amount between about 5 mA·cm⁻² and about 10 mA·cm⁻². Hence, a stable deposition of Nickel may be resulted. Finally, the mold was peeled of the structure and used for the next step. Flat polystyrene sheet was punched from cell culture Petri dish. The substrates with the cell surface pattern were obtained via a hot embossing process. Briefly, the Nickel mold flat polystyrene substrate was assembled upon each other and then pressed at about 100° C. at a pressure of about 5 MPa inside a hot press machine for about 2 minutes. After that, the Nickel mold was removed from polystyrene sheet and used for further studies.

### Example 4: Replicating the Surface Profile of Cells on a Substrate: A Photo Curing Process Technique

Replication of micro and nanostructure of PDMS mold on another substrate may be done using a photo curing process. In this example, PDMS mold was mounted on the glass slide and then was covered by a photopolymerizable resin. After blotting the excess resin, resin-covered PDMS was irradiated by UV light for about 10 min under the Nitrogen atmosphere. After completion of curing process, PDMS negative (inverse) was peeled off and as positive cured mold was obtained. This mold was used for hot embossing of other materials in similar manner as described in EXAMPLE 3.

### Example 5: Iron Oxide Nanoparticles Uptake Evaluation by the Cells Cultured on Pseudo-3D Substrates

In this example, five PDMS-based substrates produced based on the imprinted surface topography of chondrocyte cells, tenocytes cells, HeLa cell, ADSC cells and 3T6 cells according to the procedure described in EXAMPLE 2 were used here as pseudo-3D substrates for cell culturing. Then, 3T6 and HeLa cells, provided according to EXAMPLE 1, were seeded and cultured on all these five cell-imprinted PDMS substrate samples for about 3 days in order to mimic the shapes completely. Also, 3T6 and HeLa cells were seeded and cultured on a 2D flat polystyrene plate substrate (planar substrate) at the same conditions for comparison between 3D cell culturing and common 2D cell culturing. The cultured cells for each substrate were about 150,000 cells. Then, the same amount (in a range of about 100 μM to about 500 μM (about 5.7 μg/mL to about 28 μg/mL)) of fluorescence-SPIO nanoparticles as a cellular test agent were added on the top of cell-imprinted substrates seeded with cells to treat the cultured cells and the substrates were incubated to complete cells treatment and obtain treated cells with fluorescence-SPIO nanoparticles. The fluorescence-SPIO nanoparticles may have a mean core diameter of about 8 nm and a hydrodynamic diameter of about 38 nm. After about 2 hours and about 24 hours of nanoparticles addition to the cells, cells were analyzed for investigation of nanoparticles effects. To achieve accurate results, each experiment has been repeated 3 times. Cells were analyzed by cellular assays including confocal microscopy imaging, a mitochondria activity assay (MTT) assay and Immunofluorescence Microscopy.

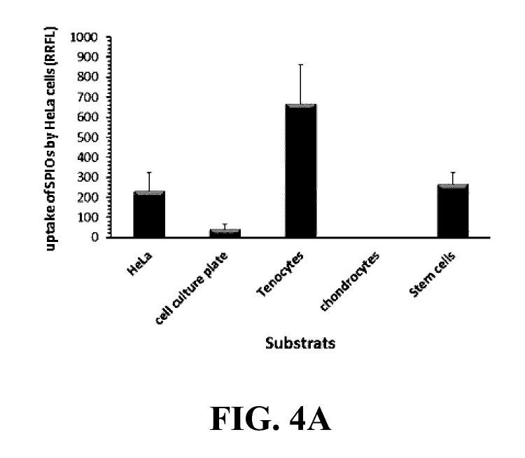

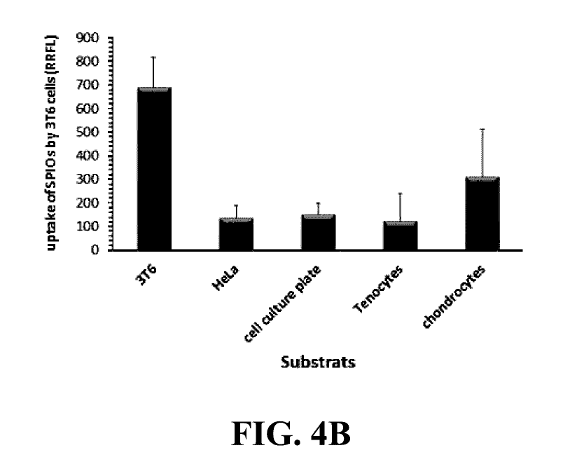

According to the confocal microscopy imaging, the uptake of SPIOs by the same cells cultured on substrates with different patterns was notably different. FIG. 4A shows the amount of SPIOs uptake (RRFL) by HeLa cells cultured on various prepared substrates having surfaces with the cell structure of HeLa, tenocytes, chondrocyte and ADSC (stem cells) cells. Moreover, the uptake of SPIOs by HeLa cells cultured on the 2D flat polystyrene plate substrate (designated by “cell culture plate”) is showed in this figure. It is observed that the SPIO uptake of the HeLa cells on flat polystyrene plate are significantly different compared to cell imprinted substrates. So, the toxicity of the SPIOs that may be induced to the cells due to a treatment by SPIOs would be significantly different depending on the cells culturing substrate surface structure or topography.

Similarly, FIG. 4B shows the amount of SPIOs uptake (RRFL) by 3T6 cells cultured on various prepared substrates having surfaces with the cell structure of 3T6, HeLa, tenocytes and chondrocyte cells. Moreover, the uptake of SPIOs by 3T6 cells cultured on a 2D flat polystyrene plate substrate (designated by “cell culture plate”) is showed in this figure. Likewise to FIG. 4A, it is observed that the surface structure of the cells culturing substrates remarkably affects to the uptake amount of a treating agent (herein, SPIOs) by the cells and consequently, the induced toxicity to the cells caused by the treating agent.

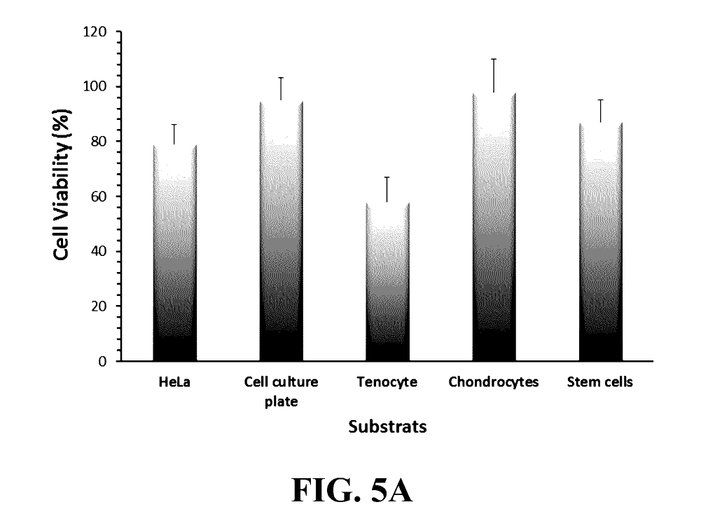

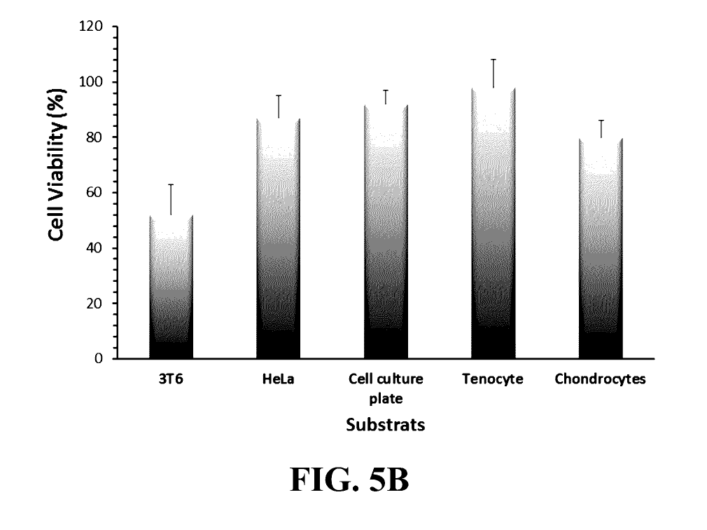

In order to probe the toxicity of SPIOs uptake on the 3T6 and HeLa cells, which were cultured on substrates with different cell shaped structures, a mitochondria activity assay was performed to determine cells viability and activity. FIG. 5A and FIG. 5B show the cell viability (%) determined by a mitochondria activity assay for the HeLa (FIG. 5A) and 3T6 (FIG. 5B) cells cultured on various substrates. It is observed that depending on the substrate, the viability of cells on flat polystyrene plate are significantly different compared to cell imprinted substrates. Also, the cells viability depends strongly to the 3D shape of the substrates surfaces. Hence, it is vital for toxicity tests to select a proper 3D culture substrate having the surface topography of the corresponding cells that should be analyzed.

In addition, an Immunofluorescence microscopy was done for further cellular analysis. Ideally, to avoid aggregation of the nanoparticles a concentration of about 500 μM was used. After about 2 hours of treatment, cells were washed in the culture medium without nanoparticles several times and cells were directly observed in microscopy. Cells were observed at a magnification of 40×, and the fluorescence signal were observed by fluorescent microscope. Total fluorescence within about 50-100 individual cells were assessed using image J software.

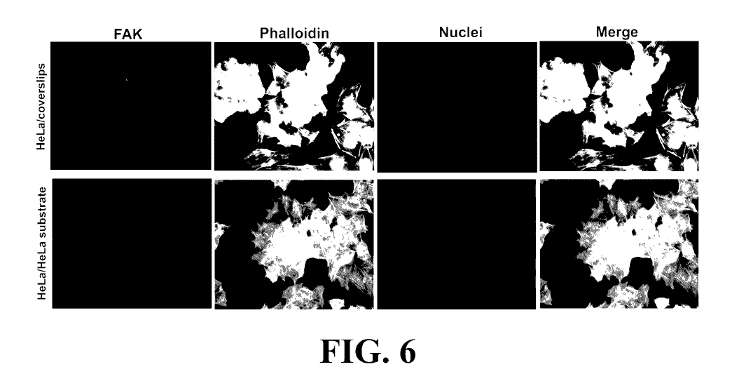

FIG. 6 shows examples of immunofluorescence microscopy images illustrating co-localization of focal adhesion kinase (FAK) with polymerized actin microfilaments in HeLa cells cultured one time on collagen-coated coverslips (cell culture plate) and another time on HeLa cells-resembling substrate and incubated with non-fluorescent nanoparticles (nanoparticles that were not coupled to Rhodamine) at a concentration of about 500 μM. Also, the Phalloidin and nuclei images of the cells are shown in FIG. 6. Furthermore, merged images representing FAK, Phalloidin and nuclei images of cells are shown for both cases. It may be observed from this figure that there is a significant difference between Immunofluorescence microscopy assay results for HeLa cells that are cultured and treated by nanoparticles on a cell culture plate (coverslips) and HeLa cells that are cultured and treated by nanoparticles on a pseudo-3D substrate having HeLa cells-resembling surface.

What is claimed is:

1. A method for cellular analysis of a plurality of biological cells, comprising:
providing a plurality of initial biological cells;
forming a plurality of cultured cells by a process comprising culturing the initial biological cells on a cell-resembling surface of a pseudo-3D substrate; and
obtaining a response of the cultured cells to at least one cell analysis assay.

2. The method of claim 1, further comprising:
evaluating the response of the cultured cells to at least one of the at least one cell analysis assays.

3. The method according to claim 1, wherein providing the plurality of initial biological cells includes:
supplying a plurality of biological cells from a cell bank or from a biopsy operation, or from a combination of a cell bank and a biopsy operation; and
culturing the plurality of biological cells to provide a plurality of biological initial cells.

4. The method according to claim 1, wherein the cell-resembling surface includes a surface having a structure resembling a surface structure of one cell, or resembling a surface structure of an association of cells, or resembling a combination of a surface structure of one cell and the surface structure of an association of cells.

5. The method according to claim 1, wherein the cell analysis assay is selected from the group consisting of gene regulation assays, gene expression assays, protein expression assays, cell proliferation assays, cell-cycle assays, cell viability assays, cell cytotoxicity assays, cell morphology assays, cell cytoskeleton assays, apoptosis assays, cell adhesion and signaling assays, cell motility assays, tissue architecture assays, co-cultures assays, biochemical assays, molecular biological assays and combinations thereof.

6. The method according to claim 1, further comprising fabricating the cell-resembling surface of the pseudo-3D substrate, wherein the fabricating includes:
forming a cell-resembling mold by a process that comprises recording the surface profile of the initial biological cells onto a first substrate; and
forming cell-resembling surface by a process that comprises replicating the surface profile of cells from the cell-resembling mold onto a second substrate.

7. The method according to claim 6, wherein the cell-resembling mold forms a pseudo-3D substrate having a cell-resembling surface.

8. The method according to claim 6, wherein the recording the surface profile of cells includes any from among imprinting, 3D imprinting, 4D printing, casting, scanning, AFM imaging, and confocal laser microscopy (CLSM).

9. The method according to claim 6, wherein the first substrate includes a crosslinkable polymer with acrylates double bonds or with epoxy bonds, and wherein the first substrate is selected from the group consisting of silicon, silicon resins and Poly(dimethylsiloxane) (PDMS).

10. The method according to claim 6, wherein the second substrate includes non-degradable polymers.

11. The method according to claim 10, wherein the second substrate includes a polystyrene (PS) or a polycarbonate substrate.

12. The method according to claim 6, wherein the replicating the surface profile of cells from the cell-resembling mold onto a second substrate includes any from among a casting process, an imaging technique, a photo curing, a hot embossing, a 3D printing, a rapid prototyping, and micro rapid prototyping using a SLA technique.

13. The method according to claim 12, wherein the hot embossing technique includes:
depositing a layer of Gold (Au) on the cell-resembling mold;
depositing a layer of Nickel (Ni) on the deposited Au layer;
forming a Nickel stamp having cells structure by a process that comprises removing the mold from the Nickel layer;
hot embossing the Nickel stamp to the second substrate; and
removing the Nickel stamp from the second substrate to obtain the pseudo-3D substrate with the cell-resembling surface.

14. The method according to claim 13, wherein the Gold layer is deposited using a sputtering technique.

15. The method according to claim 13, wherein the Nickel layer is deposited using an electroforming or electroplating technique.

16. A method for cellular analysis of a plurality of biological cells treated by a test agent, comprising:
providing a plurality of initial biological cells;
forming a plurality of cultured cells by a process that comprises culturing the initial biological cells on a cell-resembling surface of a pseudo-3D substrate;
treating the cultured cells with at least one test agent; and
obtaining a response of the treated cells to at least one cell analysis assay.

17. The method of claim 16, further comprising: evaluating the response of the treated cells to at least one of the at least one cell analysis assays.

18. The method according to claim 16, wherein the treating the cultured cells with the test agent includes:
adding the test agent on the cultured cells on the pseudo-3D substrate; and
maintaining the pseudo-3D substrate including the cultured cells with the added test agent in an incubator.
